(19) United States
(12) Patent Application Publication
(10) Pub. No.: US 2009/0076168 A1
(43) Pub. Date: Mar. 19, 2009

(54) AMINE-BASED AND IMINE-BASED POLYMERS, USES AND PREPARATION THEREOF

(76) Inventors: Tien Canh Le, Montreal (CA); Catherine Dupuis, Montreal (CA)

Correspondence Address: BERESKIN AND PARR, 40 KING STREET WEST, BOX 401, TORONTO, ON, M5H 3Y2, CA

(21) Appl. No.: 11/817,691

(22) PCT Filed: Mar. 3, 2006
(86) PCT No.: PCT/CA2006/000312

§ 371 Date: Apr. 18, 2008

Related U.S. Application Data
(60) Provisional application No. 60/658,188, filed on Mar. 4, 2005.

(51) Int. Cl. A61K 31/122; C08B 37/08; A61P 43/00; A61K 47/26

(52) U.S. Cl. 514/689; 536/20; 514/777

(57) ABSTRACT

The present invention relates to a modified polysaccharide prepared from the reaction between a polysaccharide comprising a plurality of monosaccharide subunits having at least one primary amino group, and an hydrophobic aldehyde. The aldehyde and the amino group form together an imine or amine group. The process for preparation and use in cosmetic, pharmaceutical and food industry of the modified polysaccharide is also disclosed.

### CROSS REFERENCE TO RELATED APPLICATION

This application claims the benefit of U.S. provisional application No. 60/658,188 filed Mar. 4, 2005 which is incorporated herein by reference in its entirety.

### TECHNICAL FIELD

The present invention relates to modified polysaccharide having hydrophobic functional groups attached. The invention further relates to the process for preparation and use in cosmetic, pharmaceutical and food industry of the modified polysaccharide.

### BACKGROUND OF THE INVENTION

There already exists on an international scale a significant request for aromatic components of vegetable origin. For example, vanillin, cinnamon, cuminaldehyde, etc. are used as odorous principles in certain food, essential oils (i.e. oregano, thyme, rosemary, etc.) and also consider by cosmetic or pharmaceutical industries for their disinfectants, bactericides and antiviral properties, which constitutes products of choice for hygiene and treatment of contagious diseases. Because of their very great diffusion quality, the essences can be used in external application, directly on the skin or in bath water and for treatment of internal diseases (aromatherapy). Absorbed by the skin, it quickly enters the blood circulation to be then eliminated by the lungs and the kidneys. In the process, it allows the whole organism to benefit from theirs many properties. As for their agriculture applications, the cinnamaldehyde and its derivatives (produced by Corn Rootworm Bait®) are largely used for their insecticide properties. It is also used as pesticide against acarians, mosquitoes, fungi, etc.)

It is important to mention that these aromatic substances are powerful vegetable concentrates and their use requires essential precautions. Used at too high doses, these substances can be irritating or cause allergy to skin. In certain cases, they can also cause asthma, epilepsy crises or cardiac disorder. In this context, U.S. Pat. No. 6,413,920 describes various amines that are used for releasing aldehyde or ketone perfumes over a longer period of time than by the use of the perfume itself. However, there are still various needs to be fulfilled in this field concerning the release of such compounds by means of a matrix.

In this context, the use of natural origin polymers as matrices to release these components in a controlled way is interesting. Moreover, these matrices can protect such components from oxidation or light degradation (certain components are photosensitive as for essential oils of Bergamot).

In the field of pharmaceutical compounds, the concept of controlling active ingredients release appeared in the 1930's, at the time of the attempts to add certain substance that will make it possible to decrease the active ingredient solubility in the gastric acidity. Long durations of the drug release were then observed (Dumitriu and Dumitriu, 1994). A few years later, various systems with controlled release were developed to deliver a broad range of drugs using matrices that contain polymers. One of the first systems “Ocusert®” developed by Alza Corp. (California) is still in used nowadays and is of copolymer polyethylene and polyvinyl acetate based and used, for example, to deliver pilocarpine against glaucoma. However, such synthetic polymers can have various disadvantages.

The use of natural origin polymers as matrices has several advantages: they are non-toxic, biocompatible, less expensive and easy to obtain in various forms like beads or microbeads, films (“transdermal stamps”), compressed, etc., each form being connected to the administration modes, characteristic of bioactive agents and of polymers (in term of quantity and solubility), release mechanisms and action sites. Some drugs cannot be given orally (because it cannot be absorbed via the intestinal walls) and could be encapsulated in nanoparticles for parenteral administration or entrapped in films for transdermal applications. The latter are interesting for steroids, antibiotics, analgesics, etc. release use.

In the food industry, formulations are often employed as packing or coating film and can also be employed as beads or microbeads (e.g. bacteriocine entrapment in the microbeads).

Some natural polymers and formulations thereof are often mixed with bioactive agents to entrap or immobilize them in the matrix of the polymers. To obtain a tablet, this mixture is simply put in a mould under suitable compression. Among the controlled release systems, the release mechanisms often observed are of diffusion, inflation and erosion (Peppas, L. B., Med. Plas. Biomater., 4, 34-44, 1997). The erosion or degradation control mechanism is due to the matrix slow disappearance, which progressively make it possible to release the drug in the medium. The diffusion control mechanism firstly results by the solvent access inside the support, then by the active ingredient solubilization, which allows its diffusion through the polymeric structure. The inflation release system implies several different processes. In contact with the dissolution medium, the polymers constituting the support are quickly hydrated and generate a gelled barrier (hydrogel) that gradually increases. This hydration involves a significant matrix inflation, which allows a diffusion of the active ingredient through this barrier.

Alternatively, the polymers can be so formulated as to release drugs under particular conditions. For example, some mixtures make it possible to keep tablets integrity at a neutral or basic pH, but to become soluble at an acid pH, which hydrate the tablet and releases the active ingredient. This system is often used to deliver some specific drugs in the stomach (such as Eudragit E series is a polymer formulation (butyl-methacrylate), (2-dimethyl-aminoethyl)methacrylate, methyl methacrylate and ethyl caprylate (Sheu and Rosenberg, J Food Science, 1995, 60, 98-103). Other possible aspects are also used in a microbead or microparticle forms 1—(Flick-Smith et al. Infect Immun. 2002, 70, 2022-2028) 2—(Ljeoma et al. Business Briefing Pharmatech, 2003, 203-208) so as to be usable for parenteral administrations. However, it is important to mention that some bioactive agents included in the matrix tend to diffuse through the polymeric network to migrate to the external area, which often involves a continuous loss of bioactive agents in the conservation process. In this case, the covalent immobilization of the bioactive agents on support may be interesting to prevent loss of such bioactive agents.

In view of several drawbacks with the use of synthetic polymers or polymers completely prepared form synthetic intermediates, a great interest has been shown for modified polymers obtained form natural polymers such as chitosan, alginate or cellulose, as they are non-toxic, less expensive and very abundant in nature. Moreover, the modifications brought to these polymers have permitted to confer them interesting properties. The great potential of chitosan for the monolithic systems of controlled release of drugs was reported in many documents such as U.S. Pat. No. 5,900,408. Moreover, U.S. Pat. No. 5,747,475 describes the modification of chitosan by the addition of a monosaccharide or an oligosaccharide on the C-2 level (N-glycosylation) that can be used as an additive in immunotherapy. U.S. Pat. No. 5,633,025 describes the use of carboxymethylated chitosan as a tablets coating agent. Japanese patent No. 62288602 describes the production of modified chitosan nanoparticles in order to sequester heavy metals or to entrap enzymes, etc. These nanoparticles are obtained by atomization of chitosan solution in an alkaline medium and then, by treatment of these nanoparticles in functionalization solutions as pentoxide of phosphorus, acetaldehyde or glutaraldehyde, etc.

Le-Tien et al. in WO02/094224 reported that chitosan derivation by N-acylation could confer to such a polymer a hydrophobic property, which improves resistance of polymer to water (hydrophobic-like or water-insoluble-like properties). The latter can be used as matrix for monolithic systems of controlled release by diffusion.

Films prepared from acetylated chitosan also have better mechanical properties allowing the uses of biological membrane form as transdermal or adhesive “stamps” (patch) for the mucous. Moreover, these acetylated chitosan-based films can be used as coating or packing in food protection.

Chitosan has also been studied by K. Y. Lee et al. (Blood compatibility of partially N-acylated chitosan derivatives, Biomaterials, 16, 1211-1216, 1995) by reacting it with functionalization agents such as carboxylic anhydride (i.e. acetic, propionic, n-butyric, n-valeric and n-hexanoic anhydrides). These authors reported that these derivatives are biodegradable and biocompatible. Several researchers studied the structure of acylated polymers (J Desbrieres and Al, Hydrophobic derivatives of chitosans: characterization and Theological behaviour, Int. J. Biol. Macromol. 19, 21-28, 1996) remaining in hydrophobic self-assembling.

As it can be seen the characteristics and properties of the polymers will vary according to the use which is made. However, there is still a need for a polymer which could be produced at low costs and that could be used in various applications.

### SUMMARY OF THE INVENTION

It is therefore an object of the present invention to provide a polymer which would overcome the above-mentioned drawbacks.

It is also an object of the present invention to provide a polymer which would be produced at low costs and that could be used as a matrix for the release of various active agents.

It is also an object of the present invention to provide a polymer which would be produced at low costs by using a natural polymer as starting material.

In accordance with one aspect of the present invention there is provided a modified polysaccharide resulting from the reaction between i) a polysaccharide comprising a plurality of monosaccharide subunits having at least one primary amino group, and ii) an hydrophobic aldehyde, wherein said aldehyde and said amino group form together an imine group.

According to one aspect of the invention, there is provided a modified polysaccharide resulting from the reaction between i) chitosan, and ii) between about 0.1 g to about 1 g of cinnamaldehyde or anisaldehyde for each gram of chitosan wherein said reaction between the chitosan, and the cinnamaldehyde or anisaldehyde is conducted at a pH of between about 4 to about 6.

According to one further aspect of the invention, there is provided a process for preparing a modified polysaccharide comprising i) adding a polysaccharide comprising a plurality of monosaccharide subunits having at least one primary amino group, and ii) adding a hydrophobic aldehyde, wherein said aldehyde and said amino group form together an imine group.

Still according to one further aspect of the invention, there is provided a use of a modified polysaccharide as defined herein in the manufacture of an antibacterial agent, an antifungal agent, a pesticide, a matrix for entrapping a bioactive agent, a tablet, a film, a bead, a microbead, a gel, a cream, an ointment, a lotion, a pharmaceutical formulation, a cosmetic formulation or transdermal patch.

According to one further aspect of the invention, there is provided a use of a modified polysaccharide as defined herein in the manufacture of a film for packaging and/or preserving a food product.

According to one further aspect of the invention, there is provided a method for packaging and/or preserving a food product comprising applying a film manufactured from a modified polysaccharide as defined herein 6.

According to one further aspect of the invention, there is provided a method for controlling the release of a bioactive agent comprising administering to a patient in need thereof, a formulation comprising said bioactive agent and a modified polysaccharide as defined herein in a pharmaceutically acceptable dosage.

Still according to one further aspect of the invention, there is provided a process for entrapping a bioactive agent comprising i) mixing a modified polysaccharide as defined herein and a bioactive agent, and ii) forming beads from components obtained in step i).

Still according to one further aspect of the invention, there is provided a cosmeceutical composition comprising a cosmetic agent and modified polysaccharide as defined herein.

According to one aspect of the invention, there is provided a pharmaceutical composition comprising a bioactive agent and a modified polysaccharide as defined herein.

According to one aspect of the invention, there is provided a polymer comprising a polysaccharide or an oligosaccharide which has been modified so as to include at least one imine group.

In accordance with the present invention, there is also provided a functionalized polymer having a backbone subunit of formula (I) or (II):

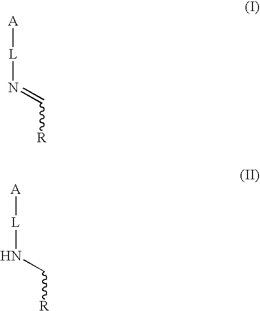

wherein:
A is a an oligosaccharide or a polysaccharide, and preferably a natural oligosaccharide or polysaccharide, and more preferably chitosan;
L is a linker or a chemical bond, and more preferably a chemical bond; and
R is an aryl-containing group having antibacterial activity, antiviral activity, antioxidant activity, antifungal activity or pesticide activity.

In one aspect, the polymer or the modified polysaccharide of the present invention can be used in the manufacture of an antibacterial agent, an antifungal agent or a pesticide.

In one aspect, the polymer or the modified polysaccharide of the present invention can be used as a matrix for entrapping a bioactive agent such as those defined herein.

In further aspects, the polymer or the modified polysaccharide of the present invention can be use in a tablet, a film, a bead, or a microbead. Alternatively, the polymer or the modified polysaccharide can be used in a gel, a cream, an ointment or a lotion, such as for the preparation of pharmaceutical formulation or a cosmetic formulation, or in the food industry. The polymer or the modified polysaccharide also finds utility in the field of agriculture.

In accordance with a further aspect of the invention, the polymer or the modified polysaccharide of the present invention can also be used as a support for a transdermal patch, or for the manufacture of such patch.

In a still further embodiment of the invention, the polymer or the modified polysaccharide can be used in the manufacture of a film for packaging a food product or for wrapping and preserving food.

Also in accordance with the present invention, there is also provided a composition comprising a polymer or the modified polysaccharide as defined herein and pharmaceutically acceptable carrier or a solvent.

Further in accordance with the present invention, there is also provided a method of preserving food, comprising the step of packaging said food with a film comprising a polymer or the modified polysaccharide as defined herein.

In a still further embodiment of the present invention, there is also provided a method of preserving food, comprising the step of packaging said food with a film comprising a polymer or the modified polysaccharide as defined herein into which a preservative agent has been entrapped so as to be released thereby preserving said food.

Further in accordance with the present invention, there is provided a process for preparing a film for packaging and/or preserving food comprising providing a solution or suspension of the modified polysaccharide as defined herein in a film forming support.

In one embodiment, the functionalized polymer or the modified polysaccharide can be used to make a film or a transdermal patch

Still in accordance with the present invention, there is also provided a process for preparing a functionalized polymer having a backbone subunit of formula (I):

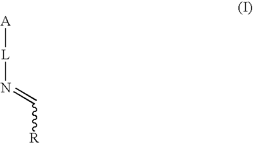

wherein:
wherein A, L and R are as defined herein.
said process comprising the step of reacting together a polymer of formula (III) and a compound (IV);

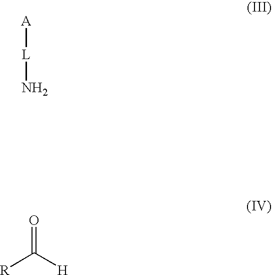

wherein A, L, and R are as previously defined.

In accordance with the present invention, the is also provided a process for preparing a functionalized polymer having a backbone subunit of formula (II):

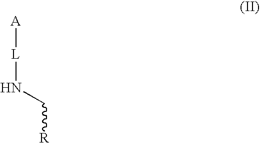

wherein:
wherein A, L and R are as defined herein;
said process comprising the step of either (i) reducing the imine group of a functionalized polymer of formula (I):

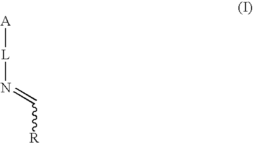

wherein A, L, and R are as previously defined to obtain the subunit of formula (II), or
(ii) reacting together an amino-substituted polysaccharide such as chitosan, an amino-substituted agarose, an amino-substituted alginate, an amino substituted pectin or an amino-substituted cellulose with an aldehyde of formula (IV)

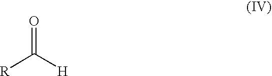

wherein R is as previously defined, to obtain the backbone subunit of formula (II).

### BRIEF DESCRIPTION OF DRAWINGS

FIG. 1 shows a comparison of FTIR spectra of a cinnamyl-chitosan polysaccharide, cinnamaldehyde and chitosan and

FIG. 2 shows release profile of acetaminophen from tablets (500 mg) based on a cinnamyl-chitosan polysaccharide (approximately 50% degree of substitution) containing 20% of drug.

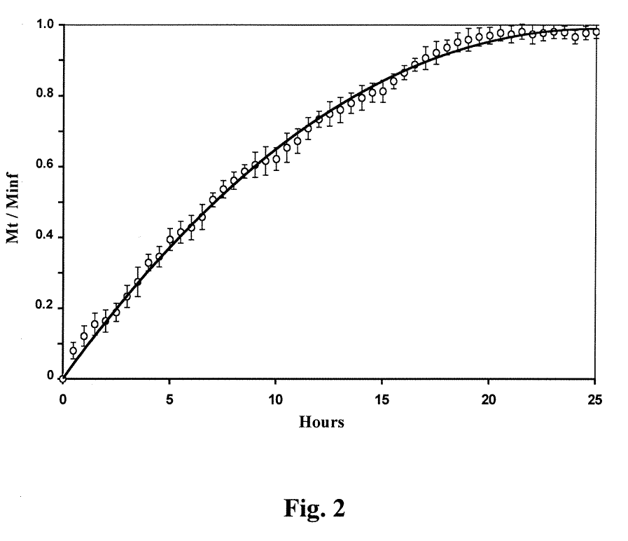

### DETAILED DESCRIPTION OF PREFERRED EMBODIMENTS

It has been found that by modifying polysaccharides such as chitosan, amino-substituted alginate, amino-substituted agarose or amino-substituted cellulose and functionalizing them with aldehydes and preferably aromatic monoaldehydes such as cinnamaldehyde, cuminaldehyde or anisaldehyde, the obtained modified polysaccharide have demonstrated interesting biological activities as antioxidant, pesticide, as well as valuable physicochemical properties allowing various applications.

In one embodiment, there is provided a modified polysaccharide resulting from the reaction between i) a polysaccharide comprising a plurality of monosaccharide subunits having at least one primary amino group, and ii) an hydrophobic aldehyde, wherein said aldehyde and said amino group form together an imine group.

In one embodiment, the hydrophobic aldehyde is selected from the group consisting of C₆aryl-C₁₋₆alkyl-CHO and C₅₋₆cyclooalkyl-CHO.

In one embodiment, the hydrophobic aldehyde is selected from the group consisting of cinnamaldehyde, methoxycinnamaldehyde, methylcinnamaldehyde, hydrocinnamaldehyde, benzaldehyde cuminaldehyde, methoxybenzaldehyde, syringaldehyde, anisaldehyde, dimethylanisaldehyde, hydroxyanisaldehyde, methylanisaldehyde, cyclohexene carboxaldehyde, myrtenal, perillaldehyde, and phellandral.

In a further embodiment, the polysaccharide is chitosan.

In a further embodiment, the polysaccharide is obtained from the reaction between agarose, alginate, pectin or cellulose and a derivatizing agent of formula X—W—NH₂, wherein X is a leaving group, W is C₁₋₁₀ alkyl.

In one embodiment, the leaving group X is selected from a chloride, a bromide an iodide.

In one embodiment, the leaving group X is a chloride.

In further embodiments:

the derivatizing agent has the formula X—W—NH₂, and W is a C1-6 alkyl;
the derivatizing agent has the formula X—W—NH₂, and W is a C1-3 alkyl;
the derivatizing agent has the formula X—W—NH₂, and W is methyl, ethyl, propyl or isopropyl;
the derivatizing agent X—W—NH₂ is 2-chloroethylamine.

In further embodiments:

at least 10% of the primary amino groups form an imine group with the aldehyde;
at least 20% of the primary amino groups form an imine group with the aldehyde;
at least 30% of the primary amino groups form an imine group with the aldehyde;
at least 40% of the primary amino groups form an imine group with the aldehyde;
between about 10% to about 90% of the primary amino groups form an imine group with the aldehyde;
between about 30% to about 80% of the primary amino groups form an imine group with the aldehyde;
between about 40% to about 50% of the primary amino groups form an imine group with the aldehyde.

In one embodiment, the reaction between said polysaccharide and said hydrophobic aldehyde is conducted at a pH of between about 3 to about 7.

In one embodiment, the reaction between said polysaccharide and said hydrophobic aldehyde is conducted at a pH of between about 4 to about 6.

In one embodiment, the reaction between said polysaccharide and said hydrophobic aldehyde is conducted at a pH of between about 4.5 and about 5.5.

In one embodiment, there is provided a modified polysaccharide resulting from the reaction between i) a polysaccharide comprising a plurality of monosaccharide subunits having at least one primary amino group, and ii) between about 0.1 g to about 1 g of an hydrophobic aldehyde for each gram of the polysaccharide.

In further embodiments:

between about 0.3 g to about 0.5 g of an hydrophobic aldehyde Is used for each gram of the polysaccharide;

about 0.4 g of an hydrophobic aldehyde is used for each gram of the polysaccharide.

In one embodiment, there is provided a modified polysaccharide resulting from the reaction between chitosan, and between about 0.1 g to about 1 g of cinnamaldehyde or anisaldehyde for each gram of chitosan wherein said reaction between the chitosan, and the cinnamaldehyde or anisaldehyde is conducted at a pH of between about 4 to about 6.

In further embodiments, modified polysaccharide resulting from the reaction between chitosan, and between about 0.1 g to about 1 g of cinnamaldehyde or anisaldehyde for each gram of chitosan

According to one embodiment of the invention, there is provided a polysaccharide or an oligosaccharide which has been modified so as to include at least one imine group.

If desired, the imine group can be reduced with a reducing agent (such as a sodium borohydride-based reagent, and more particularly such as sodium cyanoborohydride) into an amine group. In a preferred embodiment, the polysaccharide has a degree of amination of 5% to 100%.

The polysaccharide can be for example, without limitation, chitosan, an amino-substituted agarose, an amino-substituted alginate, an amino substituted pectin or an amino-substituted cellulose. When chitosan is used, said chitosan preferably has a degree of deacetylation of 60% to 100%. In another embodiment, the chitosan can also have a molecular weight of 100 to 5000 KDa.

In accordance with one embodiment of the invention, the functionalized polymer is obtained by reacting together the polysaccharide with an aldehyde, preferably a hydrophobic aldehyde and more preferably an aromatic nucleus-containing aldehyde.

In further embodiments:

the modified polysaccharide has a degree of substitution of between about 20% to about 90%;
the modified polysaccharide has a degree of substitution of between about 30% to about 80%;
the modified polysaccharide has a degree of substitution of between about 40% to about 50%.

Scheme 1 show an illustration of a modified polysaccharide being chitosan having imine groups resulting from the reaction with an aldehyde that is cinnamaldehyde.

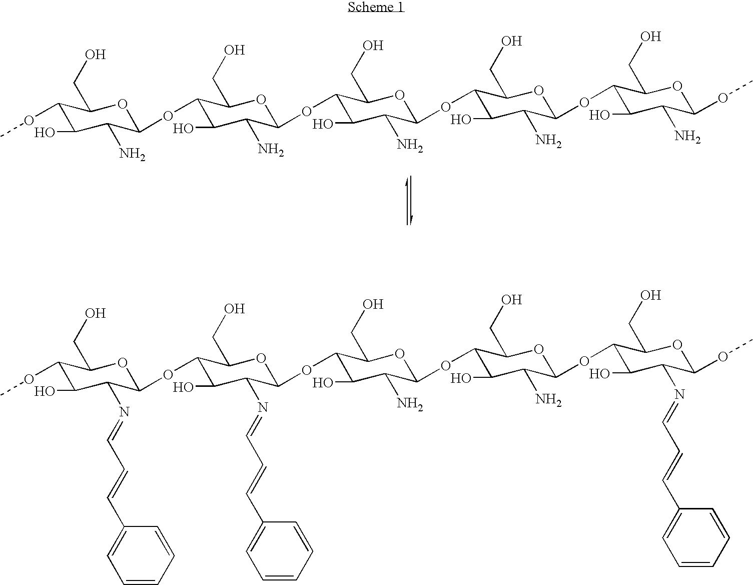

Scheme 2 illustrate a chemical reaction for reducing the imine group of the modified polysaccharide of scheme 1 using a reducing agent (such as a sodium borohydride-based reagent, and more particularly such as sodium cyanoborohydride) into an amine group.

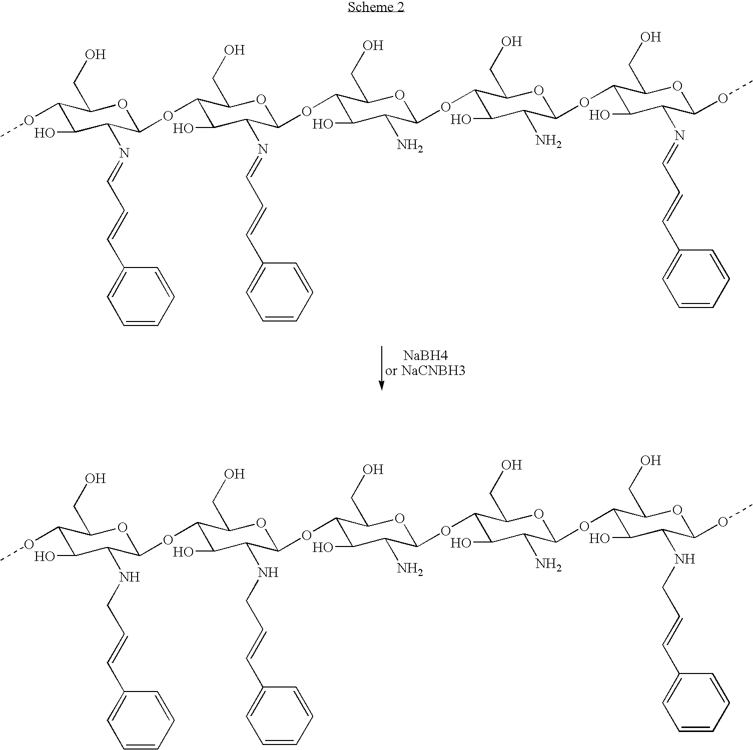

The aldehyde that can be used in accordance with the present invention can be for example selected from the group consisting of cinnamaldehyde or a derivative thereof (such as methoxycinnamaldehyde, methyl-cinnamaldehyde, and hydrocinnamaldehyde), benzaldehyde or a derivative thereof (such as cuminaldehyde, methoxybenzaldehyde, and syringaldehyde), anisaldehyde or a derivative thereof (such as dimethylanisaldehyde, hydroxyanisaldehyde, and methylanisaldehyde), and cyclohexene carboxaldehyde or a derivative thereof (such as myrtenal, perillaldehyde, and phellandral). Preferred aldehydes are those that can be an antibacterial agent, an antiviral agent, an antioxidant, an antifungal agent or a pesticide, as they have such activity.

In accordance with one embodiment of the invention, the polymer is preferably a biodegradable or biocompatible polymer.

In accordance with one embodiment of the present invention, the functionalized polymer preferably further comprising a bioactive agent immobilized therein. Such bioactive agent can be for example a drug, an enzyme, an antibacterial agent, an antifungal agent, an antioxidant, a preservative agent, a peptide or a protein, a vitamin, minerals, bacteria, or cells. Alternatively, the polymer may further comprise a preservative agent entrapped therein.

In accordance with a further embodiment, there is also provided a functionalized polymer having a backbone subunit of formula (I) or (II):

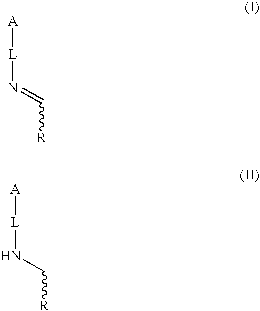

wherein:
A, L and R are as defined herein.

In one embodiment of the invention, the backbone subunit of formula (I) is obtained by reacting together a polymer of formula (III) and a compound (IV);

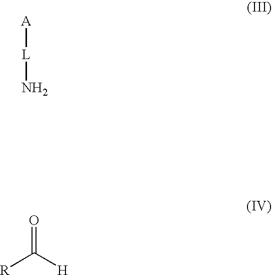

wherein A, L and R are as defined herein.

In another embodiment of the invention, the backbone subunit of formula (II) can be obtained by reducing the imine group of the backbone subunit of formula (I).

Scheme 5 illustrate the expended representation of the reaction product of alginate with 2-chloroethylamine followed by the reaction with cinnamaldehyde.

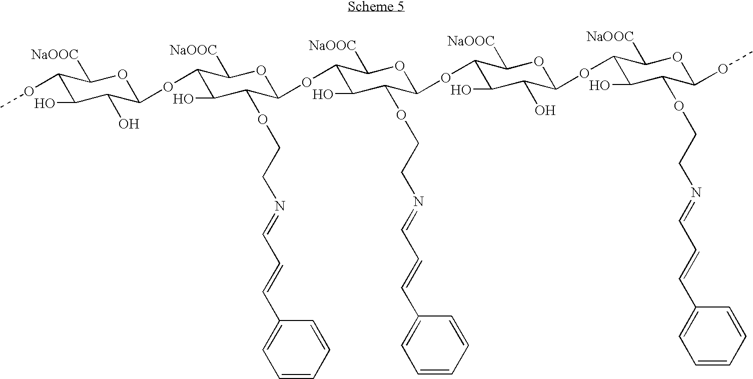

It will be understood that schemes 1 to 5 represent only particular embodiments of the present invention. As such the proportion of primary amino groups present as well as the proportion of primary amino groups forming an imine group with the aldehyde can vary In accordance with the present invention from 1 to 100%. Typically at least about 10% of the primary amino groups of the modified polysaccharide form imine, preferably at least about 20%, more preferably about 40% to about 50%.

In one embodiment, there is provided a process for preparing a modified polysaccharide comprising i) adding a polysaccharide comprising a plurality of monosaccharide subunits having at least one primary amino group, and ii) adding a hydrophobic aldehyde, wherein said aldehyde and said amino group form together an imine group.

In one embodiment, the polysaccharide is chitosan.

In one embodiment, the polysaccharide is obtained from the reaction between agarose, alginate, pectin or cellulose and a derivatizing agent of formula X—W—NH₂, wherein X is a leaving group, W is C₁₋₁₀ alkyl.

In another embodiment of the invention, A can be alginate, pectin or cellulose.

In a further embodiment of the invention, the polymer so modified has water-insoluble-like properties or is water resistant or water solubility retardant properties, depending on the modification made.

The aryl-containing group can be for example the aryl group contained in aldehydes selected from the group consisting of cinnamaldehyde or a derivative thereof (such as methoxycinnamaldehyde, methylcinnamaldehyde, and hydrocinnamaldehyde), benzaldehyde or a derivative thereof (such as cuminaldehyde, methoxybenzaldehyde, and syringaldehyde), anisaldehyde or a derivative thereof (such as dimethylanisaldehyde, hydroxyanisaldehyde, and methylanisaldehyde), and cyclohexene carboxaldehyde or a derivative thereof (such as myrtenal, perillaldehyde, and phellandral).

When the polymer or the modified polysaccharide is used as a matrix, such matrix can be used for a controlled-release of a bioactive agent, immobilized therein. Such matrix can be administered for example per os to a patient.

For the purpose of the present invention the following terms are defined below.

The term “aryl” as used herein refers to a cyclic or polycyclic aromatic ring. Preferably, the aryl group is phenyl or napthyl.

The term “heteroaryl” has used herein refers to an aromatic cyclic or fused polycyclic ring system having at least one heteroatom selected from the group consisting of N, O, and S. Preferred heteroaryl groups are furyl, thienyl, pyridyl, quinolinyl, isoquinolinyl, indolyl, isoindolyl, triazolyl, pyrrolyl, tetrazolyl, imidazolyl, pyrazolyl, oxazolyl, thiazolyl, benzofuranyl, benzothiophenyl, carbazolyl, benzoxazolyl, pyrimidinyl, benzimidazolyl, quinoxalinyl, benzothiazolyl, naphthyridinyl, isoxazolyl, isothiazolyl, purinyl, quinazolinyl, and so on.

The term “heterocyclyl” includes non-aromatic rings or ring systems that contain at least one ring having an at least one hetero atom (such as nitrogen, oxygen or sulfur). Preferably, this term includes all of the fully saturated and partially unsaturated derivatives of the above mentioned heteroaryl groups. Exemplary heterocyclic groups include pyrrolidinyl, tetrahydrofuranyl, morpholinyl, thiomorpholinyl, piperidinyl, piperazinyl, thiazolidinyl, isothiazolidinyl, and imidazolidinyl.

The terms “polysaccharide” and “oligosaccharide” as used herein are used interchangeably to refer to a molecule having a repeated monosaccharide backbone.

The term “immobilized” is used herein interchangeably with the term “entrapped”.

The expression “pharmaceutically acceptable carrier” is used herein to refer to a carrier known in the art to be acceptable in the pharmaceutical industry for an intended purpose.

The term “solvent” as used herein refers to a solvent known to the person skilled in the art for either solubilizing or brining in suspension the polymer of the present invention, in accordance with the intended use.

The term “degree of substitution” herein refers to the proportion of functionalizable amino groups that are functionalized by an aldehyde. A degree of substitution is determined using calorimetric method as described in Curofto et Aros, Anal. Biochem., (1993) 211, pp 240-241 which is hereby incorporated by reference.

The term “degree of amination” herein refers to the proportion of monosaccharide having functionalizable amino groups in a polysaccharide.

The term “alkyl” represents a linear, branched or cyclic hydrocarbon moiety having 1 to 10 carbon atoms, which may have one or more double bonds or triple bonds in the chain, and is optionally substituted. Examples include but are not limited to methyl, ethyl, propyl, isopropyl, butyl, isobutyl, sec-butyl, tert-butyl, pentyl, isopentyl, neopentyl, tert-pentyl, hexyl, isohexyl, neohexyl, allyl, vinyl, acetylenyl, ethylenyl, propenyl, isopropenyl, butenyl, isobutenyl, hexenyl, butadienyl, pentenyl, pentadienyl, hexenyl, hexadienyl, hexatrienyl, heptenyl, heptadiynyl, heptatrienyl, octenyl, octadienyl, octatrienyl, octatetraynyl, propynyl, butynyl, pentynyl, hexynyl, cyclopropyl, cyclobutyl, cyclohexenyl, cyclohex-dienyl and cyclohexyl. The term alkyl is also meant to include alkyls in which one or more hydrogen atom is replaced by a halogen, ie. an alkylhalide. Examples include but are not limited to trifluoromethyl, difluoromethyl, fluoromethyl, trifluoroethyl, difluoroethyl, fluoroethyl.

The term “optionally substituted” represents one or more halogen, amino, amidino, amido, azido, cyano, guanido, hydroxyl, nitro, nitroso, urea, OS(O)₂Rm (wherein Rm is selected from C₁₋₆ alkyl, C₆₋₁₀ aryl or 3-10 membered heterocycle), OS(O)₂ORₙ (wherein Rₙ is selected from H, C₁₋₆ alkyl, C₆₋₁₀ aryl or 3-10 membered heterocycle), S(O)₂ORₚ (wherein Rₚ is selected from H, C₁₋₆ alkyl, C₆₋₁₀ aryl and 3-10 membered heterocycle), S(O)₀₋₂R_{q} (wherein R_{q} is selected from H, C₁₋₆ alkyl, C₆₋₁₀ aryl or 3-10 membered heterocycle), OP(O)ORₛORₜ, P(O)ORₛORₜ (wherein Rₛ and Rₜ are each independently selected from H or C₁₋₆ alkyl), C₁₋₆alkyl, C₆₋₁₂aralkyl, C₆₋₁₀aryl, C₁₋₆alkoxy, C₆₋₁₂aralkyloxy, C₆₋₁₀aryloxy, 3-10 membered heterocycle, C(O)Rᵤ, (wherein Rᵤ is selected from H, C₁₋₆ alkyl, C₆₋₁₀ aryl, C₆₋₁₂ aralkyl or 3-10 membered heterocycle), C(O)ORᵥ, (wherein Rv is selected from H, C₁₋₆ alkyl, C₆₋₁₀ aryl, C₆₋₁₂ aralkyl or 3-10 membered heterocycle), NRₓC(O)_{Rw} (wherein Rx is H or C₁₋₆ alkyl and R_{w} is selected from H, C₁₋₆ alkyl, C₆₋₁₀ aryl, C₆₋₁₂ aralkyl or 3-10 membered heterocycle, or Rₓ and R_{w} are taken together with the atoms to which they are attached to form a 3 to 10 membered heterocycle) or SO₂NR_{y}R_{z}, (wherein R_{y} and R_{z} are each independently selected from H, C₁₋₆ alkyl, C₆₋₁₀ aryl, C₃₋₁₀ heterocycle or C₆₋₁₂ aralkyl).

The term “leaving group” herein refers to an atom or molecule that detaches from the group C₁₋₁₀alkyl when exposed to an hydroxyl group of a monosaccharide under usual reaction conditions. Examples include halogens such as chloride, bromide and iodide, sulfonates such as trifluoromethanesulfonate and methanesulfonate, azide.

The term “hydrophobic aldehyde” herein refers to the physical property of an aldehyde that is repelled by water. Examples of such aldehydes include aldehydes such as C₆aryl-C₁₋₆alkyl-CHO and C₅₋₆cyclooalkyl-CHO. The aryl and alkyl are optionally substituted. Examples include without limitation cinnamaldehyde or a derivative thereof (such as methoxycinnamaldehyde, methyl-cinnamaldehyde, and hydrocinnamaldehyde), benzaldehyde or a derivative thereof (such as cuminaldehyde, methoxybenzaldehyde, and syringaldehyde), anisaldehyde or a derivative thereof (such as dimethylanisaldehyde, hydroxyanisaldehyde, and methylanisaldehyde), and cyclohexene carboxaldehyde or a derivative thereof (such as myrtenal, perillaldehyde, and phellandral).

The term “organic acid” herein refers to an organic compound that has carboxylic (—COOH) or sulfonic group (—SO3H). Examples include without limitation carboxylic acids such as formic acid, acetic acid, chloroacetic acid, and sulfonic acid such as methanesulfonic acid and ethanesulfonic acid.

The term “bioactive agent” herein refers to drug, an enzyme, an antibacterial agent, an antifungal agent, an antioxidant, a preservative agent, a peptide or a protein, a vitamin, minerals, bacteria, or cells.

“Oral dosage” may conveniently be presented as discrete units such as capsules, cachets or tablets each containing a predetermined amount of the active ingredient; as a powder or granules; as a solution, a suspension or as an emulsion. Tablets and capsules for oral administration may contain conventional excipients such as binding agents, fillers, lubricants, disintegrants, or wetting agents. The tablets may be coated according to methods well known in the art. Oral liquid preparations may be in the form of, for example, aqueous or oily suspensions, solutions, emulsions, syrups or elixirs, or may be presented as a dry product for constitution with water or other suitable vehicle before use. Such liquid preparations may contain conventional additives such as suspending agents, emulsifying agents, non-aqueous vehicles (which may include edible oils), or preservatives.

“Transdermal dosage” may be presented as ointments, creams or lotions, or as a transdermal patch. Such transdermal patches may contain penetration enhancers such as linalool, carvacrol, thymol, citral, menthol and t-anethole. Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or colouring agents.

The term “reducing agent” herein refers to an agent able to reduce an imine into an amine without detrimental effect on the polysaccharide. Preferred agent include hydride reducing agents. Typical hydride reducing agents include aluminium-based agent such as lithium aluminium hydride (LiAlH₄), aluminium hydride (AlH₃), boron-based agent such as sodium borohydride (NaBH₄), sodium cyanoborohydride (NaCNBH₃).

Alternatively to reducing agents, reducing systems such as electrochemical cells may also be used under proper reductive conditions.

The term “film recovery rate” herein refers to the recovery (i.e. weight after vs weight before) of a film, according to the present invention, when the film is left to soak in water for 24 hours following the protocole described in Le Tien et al. (J. Agric. Food Chem. 2000, 48, 5566-5575) that is herein incorporated by reference in entirety.

Functionalization of an polysaccharide with an aldehyde can confer to polymers or the modified polysaccharides not only good rheological properties (hydrophobicity due to interactions of the functionalized aromatic rings between the two macromolecular chains (see scheme 6), but also of the biological activities already quoted above. For example, chitosan functionalized with cinnamaldehyde can provide an active polymer which is more hydrophobic while keeping a biological activity.

As shown in scheme 3, the intermolecular interactions between the phenyl groups generate hydrophobic interaction.

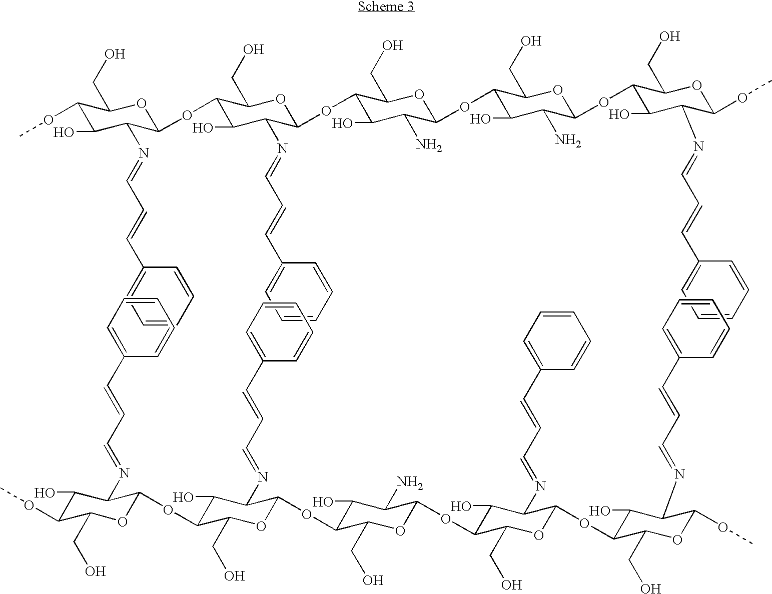

Also, chemical groups formed from the covalent bonding between the polymers and aldehydes following the functionalization are generally imine (bases of Schiff), hemi-acetal or acetal groups. In this context, these bonds are reversible since they can be hydrolyzed and the bioactive agents (aldehydes) can be released in a controlled way when they are in contact with a dissolution medium.

The formation of imine groups can be achieved by functionalization of chitosan amine groupings with aldehyde (C-2). However, hemiacetals or acetals are possibly formed with hydroxyl groups of polymers such as alginate, agarose, cellulose, pectin, chitosan etc.

In another aspect, hydroxyl groups of polymers or polysaccharides (such as alginate, cellulose, pectin, amylose, agarose) can be modified so as to include amine groups. They are reacted with an amino-based reagent which also acts as a linker. As an example, polysaccharides can be reacted with alkylamine chlorides (such as chloroethylamine) so as to acquire amine groups. Then, they can functionalized with aldehydes via the formation of imine bonds. The length of the alkyl chain can vary so as to make it possible to outdistance the polymer and bioactive agent. The linker thus also acts as a spacer. This role can be very interesting so as to facilitate bioactive agents access of substrates or to improve the polymer physico-chemical properties for some specific applications.

Scheme 4 illustrate the chemical derivatization of a monosaccharide subunit c of a polysaccharide that is alginate with a derivatizing agent (X—W—NH₂) that is 2-chloroethylamine followed by the reaction with an aldehyde that is cinnamaldehyde.

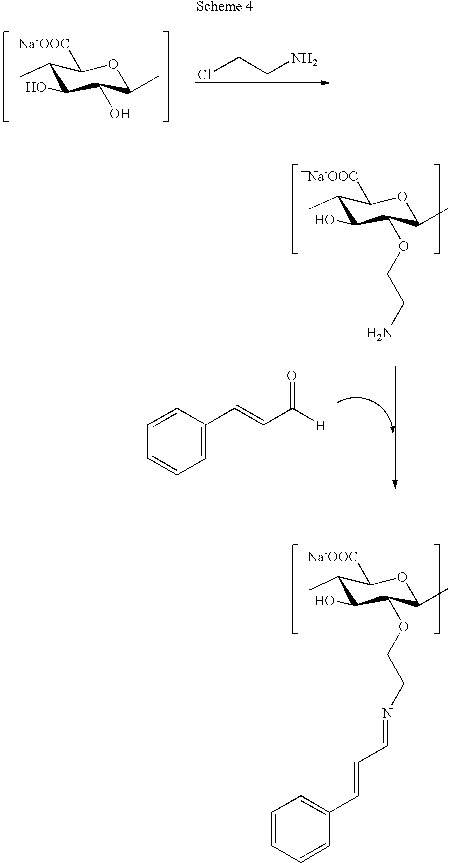

In one embodiment, the process further comprise the step of reducing imine groups to amine groups.

In further embodiments:

the reduction is conducted using a hydride reducing agent,
the reduction is conducted using a boron-based hydride reducing agent, the boron-based hydride reducing agent is sodium borohydride (NaBH₄) or sodium cyanoborohydride (NaCNBH₃),
the reduction is conducted using a aluminum-based hydride reducing agent,
the aluminum-based hydride reducing agent lithium aluminium hydride (LiAlH₄), aluminium hydride (AlH₃).

It will also be understood that derivatizing agent (X—W—NH₂) such as 2-chloroethylamine, may react with the hydroxyl groups at any carbon of the monosaccharide subunit. Scheme 6 illustrate without limitation some examples.

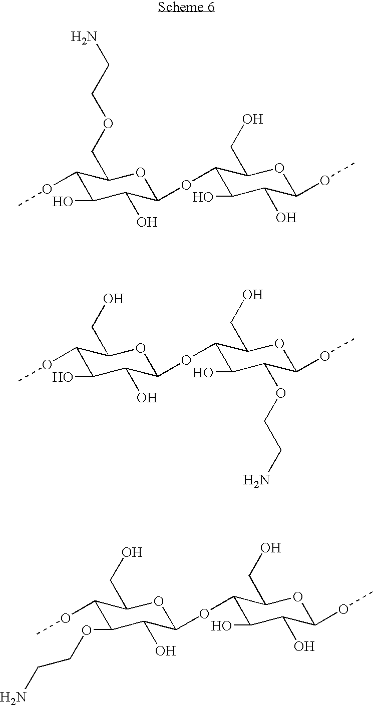

The bioactive agents can be defined as agents having an effect on a biological system. It can be drugs, nutraceutics (vitamins and minerals), probiotics (lactic bacteria), enzymes, peptides (bacteriocines), antioxidants or antimicrobial.

For the pharmaceutical applications, the polymers or the modified polysaccharide as defined herein can be used as supports for active ingredients release. For this purpose, the functionalization with aromatic monoaldehydes confers better rheological properties with a sufficient hydrophobicity degree (due to the aromatic rings). These polymers or the modified polysaccharides can be obtained in powder for tablets manufacturing (the most used form due to its simplicity and economy) by direct compression. Moreover, the administration way of this form is primarily “per os” (oral way), which is consider as the most natural, simplest and sedentary way. The tablets manufacturing by direct compression consists of a mechanically mixing of a drug with an adequate polymeric support and by compressing the mixture under suitable pressure.

The release mechanism could be based on diffusion or inflation followed by the diffusion of the active compound. Also, these matrices could be used in other forms such as beads, microbeads or nanoparticles and the administration could be carried out respectively by oral or parenteral way.

In one embodiment, there is provided the use of a modified polysaccharide as defined herein in the manufacture of any one of the following applications: an antibacterial agent, an antifungal agent, a pesticide, a matrix for entrapping a bioactive agent, a tablet, a film, a bead, a microbead, a gel, a cream, an ointment, a lotion, a pharmaceutical formulation, a cosmetic formulation or transdermal patch.

In a further embodiment, there is provided a method for controlling the release of a bioactive agent comprising administering to a patient in need thereof, a formulation comprising said bioactive agent and a modified polysaccharide as defined herein in a pharmaceutically acceptable dosage.

In a further embodiment, the dosage is a transdermal dosage.

In a further embodiment, the dosage is an oral dosage.

A other aspect of this invention is that the functionalization agents used are hydrophobic monoaldehydes and preferably aromatic monoaldehydes.

For the food applications such as coating or packing, the films containing these obtained polymers following functionalization are not only resistant to water, but also have antioxidant activities. Consequently, they are very interesting to use for food preservation for a long period while preserving their physicochemical quality. Moreover, they make it possible to protect food against oxidation or contamination from pathogenic bacteria.

In one embodiment, there is provided the use of a modified polysaccharide as defined herein in the manufacture of a film for packaging and/or preserving a food product.

Further in accordance with one embodiment, there is provided a process for preparing a film for packaging and/or preserving food comprising providing a solution or suspension of the modified polysaccharide, as defined herein, in a film forming support and substantially drying said film.

In one embodiment, the film is prepared at about room temperature.

In one embodiment, the process further comprise the step of adding a gelling agent.

In one embodiment, therefore a film for packaging and/or preserving a food product comprising a modified polysaccharide as defined herein is provided.

In further embodiment:

the film has a film recovery rate of at least 30%;
the film has a film recovery rate of at least 40%;
the film has a film recovery rate of at least 50%;
the film has a film recovery rate of at least 60%.

Further in accordance with the present invention, there is also provided a method of preserving food, comprising the step of packaging said food with a film comprising a polymer or the modified polysaccharide as defined herein.

In one embodiment, the aldehyde is released over a predetermined period of time. Alternatively, the polymer can be hydrolysable at a pH of about 3.5 to about 5.0, so as to release the aldehyde or the bioactive agent.

With regard to the agriculture field, these modified polymers can be used as pesticides. It is interesting to mention that as for the chitosan functionalization with trans-cinnamaldehyde, the obtained product has several advantages and interesting properties:
A natural, non-toxic and biodegradable pesticide;
A weak loss of bioactive agents (trans-cinnamaldehyde i.e.), which are covalently linked to polymer;
A controlled release bioactive agents (trans-cinnamaldehyde i.e.);
A pesticide activity (trans-cinnamaldehyde i.e.);
A stimulating activity for the resistance system against pathogenic plants' bacteria due to the chitosan.

Consequently, a modified polysaccharide (such as chitosan or alginate) with aromatic aldehydes functionalization helps to acquire several different properties (hydrophobic subject, antibacterial, antiacarial and pesticides, etc.)

The use of functionalized polymers as matrices presents several advantages:
1. Derivation with aromatic aldehydes can limit the water access in the matrix, which involves a long release controlled by diffusion;
2. The aromatic can interact between them via the hydrophobic interactions and thus improving the mechanical properties of the matrix;
3. Aromatic monoaldehydes used as the functionalization agents can have interesting biological activities (antifungic, pesticidal, etc.) and consequently, the functionalized matrix can acquire these properties after functionalization;
4. The matrix can protect the bioactive agents in denaturing medium;
5. The matrix can be obtained in several forms: beads, microbeads, tablets, implants, gel, films, etc. allowing to increase the field application.

The chitosan and alginate are preferably used as matrices. The chitosan is obtained from chitin after deacetylation whose repetitive monomeric unity is primarily of glucose-2-amine. Generally, it is on the C-2 amine groups that the functionalization takes place (Oyrton and Claudio, Int. J Biol Macromol, 26, 119-128, 1999).

In one embodiment, there is provided a process for entrapping a bioactive agent comprising i) mixing a modified polysaccharide as defined herein and a bioactive agent, and ii) forming beads from components obtained in step i).

In a further embodiment, there is provided a cosmeceutical composition comprising a cosmetic agent and modified polysaccharide as defined herein.

The cosmetic agents for use in the present invention are not particularly limited. Exemplary cosmetic agents are described in C.T.F.A. Cosmetic Ingredient Handbook, First Edition, 1988, which is hereby incorporated by reference.

In still a further embodiment, there is provided a pharmaceutical composition comprising a bioactive agent and a modified polysaccharide as defined herein.

The bioactive agent for use in the present invention are not particularly limited. Exemplary drugs used as bioactive agent are described in Physicians Desk Reference, 2005 ed, Thomson which is hereby incorporated by reference.

The alginate is a polysaccharide product from Phaeophyceae algae. It is composed of alternative sequences of two acids, B-D-mannuronic (BETA) and α-L-glucuronic (Haug, Rept. No 30, Norwegian Institute Seaweed Research, Trondheim, Norway, 1964). The alginate can be modified by different methods whose direct functionalization is carried out between hydroxyl and aldehyde groups to form hemi-acetals or acetals. For the indirect functionalization, a preliminary coupling to alginate with alkylamine chlorides is necessary. It is significant to mention that polysaccharides as amylose, cellulose, carragenane, agarose, hyaluronane, etc. can be modified as described for alginate.

Although aromatic imines are more stable than aliphatic imines, it is possible to stabilize the imine-containing compound by reducing the imine bond with sodium borohydride. Thus, for example, cinnamylamine chitosan is more stable than its corresponding imine, cinnamylimine chitosan. Using these properties, it may in some case be desirable that the compound be less stable in the form of the imine-containing compound, such that the compound is readily released from the matrix or the polymer. This thus allows for the release of bioactive agents, as is often desired in the agrifood industry. However, in the pharmaceutical industry, it is more often desired that the compound be more stable so as to delay it degradation, allowing for a slow release of the bioactive compound (i.e nisin) from the matrix. Therefore, with the teaching that the amine-containing compound is more stable than its corresponding imine-containing compound, one skilled in the art will choose the amine or imine-containing compound depending on the desired use.

The present invention will be more readily understood by referring to the following examples which are given to illustrate the invention rather than to limit its scope.

### EXAMPLE 1

### Modified Polysaccharide with Trans-Cinnamaldehyde for Use as an Emulsifying Agent of Essential Oils

### Cinnamyl Chitosan Synthesis

A chitosan quantity of 5 g was dissolved in 600 mL of organic acid solution (preferably acetic acid, 0.2 M). When the chitosan was completely solubilized, the pH of solution was adjusted at 4.5-5.5 with NaOH 0.1 M and different volumes (0.5-5.0 mL) of cinnamaldehyde was slowly added to obtain various degrees of substitution. The reaction is carried out at 40-60° C. during 3 to 48 hours and finally the functionalized chitosan solution was thereafter to 1 L with distilled water.

### Cinnamyl Alginate Synthesis

The modified alginate synthesis can be done the same way as for chitosan. However, a preliminary derivation (scheme 2) was also interesting to confer to alginate more reactive amine groupings. For alginate aminoethylation, 5 g of sodium alginate were dispersed in 400 ml of 1.0-1.2 M NaOH solution and kept at room temperature for 2 h for swelling. The solution was heated to 70° C. and then 20-80 g of 2-chloroethylamine hydrochloride, dissolved in a minimal volume (50-100 ml) of water just prior to synthesis, were added. The reaction was allowed to continue for 1 h at 70° C. and the product washed and dried to obtain the powder.

### Essential Oil Emulsification

An essential oil volume of 1-10 mL (thyme, oregano or rosemary) was added to 100 mL of functionalized polymer solution and stirring at high speed for at least 30 minutes (using “ultra-turex” preferably). The solution was stable and no phase separation was observed. Without being bound to theory, it is believed to be caused by the hydrophobic interactions between aromatic rings of essential oil and cinnamyl residues.

### EXAMPLE 2

### Films Formulation Containing Chitosan Modified with Aldehydes

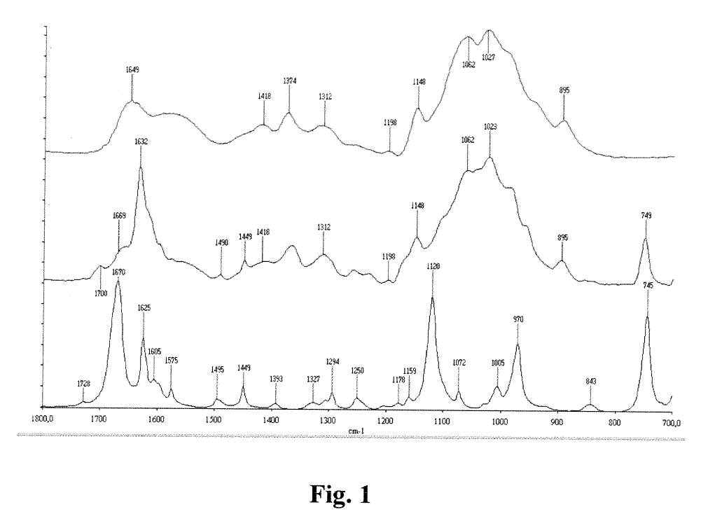

A chitosan quantity of 5.0 g was dissolved in 600 mL of lactic acid solution of 0.2 M. After homogenizing, a volume between 0.5-5.0 mL of cinnamaldehyde (or benzaldehyde or anisaldehyde) was added drop by drop. The reaction was allowed to continue for at least 3 hours at 60-80° C. with stirring. The glycerol addition (0.1-10%) to improve the mechanical properties (in particular viscoelasticity) is optional. The solution was completed to 1 L then distributed (20-40 mL) in Petri boxes and dried at room temperature for 24-48 h. Just before film was completely dried, the addition of gelling agent (i.e polyphosphate salts or sodium hydroxycitrate providing Garcinia cambogia) was possible in order to increase the water resistance of film. The films were separated for FTIR analysis and preserved at 54% of relative humidity for at least 24 hours before rheological tests. The FTIR analysis of FIG. 1 was obtained using Spectrum One-UATR (Universal Attenuated Total Reflectance).

The mechanical properties analysis was carried out with the texturometer of Stevens LFRA type (Analyzer Texture, model TA/1000, Scarsdale, N.Y.) and the solubility films test was carried out as described by Le Tien et al. (J. Agric. Food Chem. 2000, 48, 5566-5575, 2002). Film thickness was measured using a Mitutoyo Digimatic Indicator (Mitutoyo, Tokyo, Japan) at five random positions around the film. The average film thickness was in the range of 50-60 μm.

As for the native chitosan films, the puncture strength was approximately 520 N/mm, but no elasticity was observed. For the solubility test, the recovery rate (carried at 22° C.) was 5% suggesting a great film solubility of the native based chitosan. Thus, the chitosan functionalization with cinnamidehyde gave to the film a higher hydrophobicity whose recovery rate was 61%. Without being bound to theory, it is believed that this phenomenon is due to hydrophobic interactions of the cinnamaldehyde aromatic ring between the two macromolecular chains (as described in scheme 6). Moreover, the film had a viscoelasticity coefficient of about 0.75 suggesting that the functionalization makes the film more elastic. This elasticity could be due to the presence of adjacent cinnamyl residues of the chitosan chains. These residues interact between them (hydrophobic interactions), which decrease the hydrogen interactions and increases the film flexibility by reacting as a plasticizing agent. However, its puncture strength was decreased to 180 N/mm value, but it was sufficiently rigid for food applications such as coating and packing purposes. It is valuable to note that the recovery rate of the modified chitosan films was higher (about 60%), which indicates a water resistance and could be used as direct contact packing such as for packaging humid food like meat, fruits, vegetables, etc.

### EXAMPLE 3

### Modified Chitosan-Based Creams or Lotions Formulation to Simultaneously Entrap the Bioactive Agents of Hydrophobic Nature (CoQ10) and of Hydrophilic Nature (Vitamin C)

The matrix preparation for creams was the same as described in the example 1 for cinnamyl chitosan synthesis.

To obtain the powder, the solution was precipitated in ethanol and dried with acetone. Spray-drying could also be used.

For the cream formulation, the components as described in table 1 can be mixed in a flask. The cream formulation was also prepared as described in Table 2.

TABLE 1

| Component | Qty(% w/w) |
|---|---|
| Aquous solution: | |
| Functionalized chitosan | 1-2% |
| Vitamin C | 5-10% |
| Hyaluronate (optional) | 0.5-1% |
| Water | 60-80% |
| Oily solution | |
| Vegetal oil (preferably canola oil) | 1.-5.% |
| Shea butter (or polawaxtm) | 5-12 % |
| CoQ10 | 2-5% |
| Tween ™ (20-100) or Miglyol ™ | 0.5-5% |

TABLE 2

| Component | Qty (% w/w) |
|---|---|
| Aquous solution: | |
| Functionalized chitosan | 2% |
| Vitamin C | 7% |
| Hyaluronate (optional) | 1% |
| Water | 70% |
| Oily solution | |
| Vegetal oil (preferably canola oil) | 5% |
| Shea buffer (or polawaxtm) | 5% |
| CoQ10 | 5% |
| Tweentm (20-100) or Miglyoltm | 5% |

The % being expressed in relation with the total weight of the solution. The amount of each component can be modified from those described, and the total to 100% adjusted with water and/or vegetal oil (for example, when the lower amount in the range is used).

For aqueous solution, the modified chitosan was homogenized for 30 min-2 h at 60° C. For the oily solution, the CoQ10 was dissolved in vegetable oil, shea butters and Tween™ at the same temperature (60° C.). The cream was obtained after mixing two solutions with moderate agitation until a uniform suspension was obtain. The addition of Polawax™ (2-4%), cetyl alcohol (2-4%), fatty acids (stearic or palmitic acid, 2-5%) and Tween 20% are optional to obtain the desired texture. The obtained cream was cooled to room temperature and had an approximate viscosity of 400-1000 cps.

### EXAMPLE 4

### Modified Chitosan Beads Formulation for Entrapment of Bioactive Agents: Vitamin B6 (Pyridoxine)

An amount of modified chitosan of 1.0-2.0% synthetized as described in example 1 was dissolved in acetic acid solution (0.1 M) and the pH adjusted between 4.5-5.5. An amount of vitamin was then dispersed in solution under agitation. This mixture was then introduced into a syringe with a suitable diameter needle and left to drain off in a polyphosphate salts (5-10%) solution to obtain the beads.

The microbeads can also be obtained by atomization of mixture in gelation solution. The mixture (functionalized chitosan/vitamin B6) was then decanted by sedimentation.

The required amount of chitosan used to form beads was varied depending on the chitosan molecular weight. For instance 1.0-1.5% (w/w) of polysaccharide was used for chitosan 500-600 kDa. About 2.5% of polysaccharide was used for chitosan 150-300 kDa. The desired mechanical properties of the beads may therefore be adjusted by varying the molecular weight. Preferably, chitosan having a molecular weight of 500-600 kDa at concentration of about 1.5% and a degree of substitution of about 20% is used.

### EXAMPLE 5

### Use of Modified Chitosan as a Support for Transdermal Release of Bioactive Agents

The modified chitosan was synthesized as previously described in the example 1 for cinnamyl chitosan synthesis, but with different substitution degrees of about 20-80% and the pH solution adjusted to 4.5-5.5 with NaOH (0.1 M). Bioactive molecules can be added and the mixture stirred for 30 minutes to 2 hours according to their liposoluble or water-soluble nature. The addition of collagen or gelatin (1-10%), Polawax™ (1-10%), cetyl alcohol (1-10%), fatty acids (stearic or palmitique, 1-10%), Tween™ 20% and terpenoid (i.e. limonene) is optional.

The results with the guaranine (50 mg/mL) of this study showed that there was a penetration of the bioactive agent after 1 hour of treatment by application of 1-2 mL solution or gel/100 cm² of skin to the left forearm. This penetration resulted in the observation of manifestations that were a considerable increase of blood pressure and pulsations on young subjects and without preliminary use of drugs or stimulating substances compared to the untreated subjects.

The addition of other substances in the solution, like essential oils, Cat's Claw, Capsacin, etc. can be interesting so as to improve and increase penetration (permeation agents) of the bioactive agents.

### EXAMPLE 6

### Use of Functionalized Chitosan as Supports for Controlled Liberation System by oral Administration

Chitosan has been modified as previously described in the example 1 and with different substitution degrees of about 10-50%. The latter was then precipitated in acetone then rewashed in the same solvent 3 times to obtain the corresponding powder. Tablets of 500 mg of functionalized chitosan with cuminaldehyde containing 20% of acetaminophen as tracer were tested in an aqueous medium (pH 7.0-7.2, 50 rpm) with Distek appliance using USP XXVII method. For the native chitosan, the content was quickly released within 1 hour. However, the modified chitosan (40-50% substitution degree) based tablets were released of their contents (t₉₀) only after 12-18 h.

### EXAMPLE 7

### Reduction of Imine Groups Using Borohydride Source

The imine function of the modified polysaccharide may be reduced to the amine using a reducing agent such as sodium cyano borohydride or sodium borohydride. The alcoholic solution (or suspension) of the imine containing modified polysaccharide is treated with about 1 equivalent of the reducing agent per imine function at about zero degree celcius to room temperature. Alternatively, less reducing agent may be used depending on the conditions used. When the reduction is completed, the unreacted reducing agent is treated and the amine modified polysaccharide is extracted from the reaction medium using standard isolation procedures. The modified polysaccharide is optionally purified using standard purification procedures.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

1. A modified polysaccharide resulting from the reaction between
i) a polysaccharide comprising a plurality of monosaccharide subunits having at least one primary amino group, and
ii) an hydrophobic aldehyde,
wherein said polysaccharide is chitosan, and wherein about 10% to about 90% of said primary amino groups form an imine group with said aldehyde.

2. The modified polysaccharide of claim 1, wherein said hydrophobic aldehyde is selected from the group consisting of C₆aryl-C₁₋₆alkyl-CHO and C₅₋₆cyclooalkyl-CHO.

3. The modified polysaccharide of claim 1, wherein said hydrophobic aldehyde is selected from the group consisting of cinnamaldehyde, methoxycinnamaldehyde, methyl-cinnamaldehyde, hydrocinnamaldehyde, benzaldehyde, cuminaldehyde, methoxybenzaldehyde, syringaldehyde, anisaldehyde, dimethylanisaldehyde, hydroxyanisaldehyde, methylanisaldehyde, cyclohexene carboxaldehyde, myrtenal, perillaldehyde, and phellandral.

4. (canceled)

5. (canceled)

6. (canceled)

7. (canceled)

8. (canceled)

9. (canceled)

10. (canceled)

11. (canceled)

12. The modified polysaccharide of claim 1, wherein about 30% to about 80% of said primary amino groups form an imine group with said aldehyde.

13. The modified polysaccharide of claim 1, wherein about 40% to about 50% of said primary amino groups form an imine group with said aldehyde.

14. The modified polysaccharide of claim 1, wherein the reaction between said polysaccharide and said hydrophobic aldehyde is conducted at a pH of between about 4 to about 6.

15. The modified polysaccharide of claim 14, wherein the pH is between about 4.5 and about 5.5.

16. A modified polysaccharide resulting from the reaction between:
i) chitosan, and
ii) about 0.1 g to about 1 g of cinnamaldehyde or anisaldehyde for each gram of chitosan,
wherein said reaction between the chitosan, and the cinnamaldehyde or anisaldehyde is conducted at a pH of between about 4 to about 6.

17. (canceled)

18. (canceled)

19. (canceled)

20. (canceled)

21. (canceled)

22. (canceled)

23. A method for controlling the release of a bioactive agent comprising administering to a patient in need thereof, a formulation comprising said bioactive agent and a modified polysaccharide as defined in claim 1 in a pharmaceutically acceptable dosage.

24. The method as defined in claim 23, wherein said dosage is a transdermal dosage.

25. (canceled)

26. (canceled)

27. A cosmeceutical composition comprising a cosmetic agent and a modified polysaccharide as defined in claim 1.

28. A pharmaceutical composition comprising a bioactive agent and a modified polysaccharide as defined in claim 1.

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. The modified polysaccharide as defined in claim 1, wherein said hydrophobic aldehyde is cinnamaldehyde.

64. A transdermal dosage form comprising a modified polysaccharide as defined in claim 1, and a bioactive agent.

65. The transdermal dosage form of claim 64, wherein said dosage form is a gel or a cream.

66. The transdermal dosage form of claim 64, wherein said dosage form is a transdermal patch.

67. The transdermal dosage form of claim 64, wherein said bioactive agent is CoQ10.

68. A method of using a modified polysaccharide as defined in claim 1, said method comprising entrapping a bioactive agent into a matrix comprising said modified polysaccharide so as to permit a controlled release of said bioactive agent.

69. A modified polysaccharide resulting from the reaction between
i) a polysaccharide comprising a plurality of monosaccharide subunits having at least one primary amino group, and
ii) an hydrophobic aldehyde,
wherein said polysaccharide is obtained from the reaction between agarose, alginate, pectin or cellulose and a derivatizing agent of formula X—W—NH₂, wherein X is a leaving group, W is C₁₋₁₀ alkyl, and wherein said aldehyde and said amino group form together an imine group.
